(19) United States
(12) Patent Application Publication
(10) Pub. No.: US 2004/0138302 A1
(43) Pub. Date: Jul. 15, 2004

(54) Antifungal antibiotics

(75) Inventors: Ping Cai, New City, NY (US); Fangming Kong, River Vale, NJ (US); Pamela Fink Charbonneau, New City, NY (US); Mark Edward Ruppen, Garnerville, NY (US)

Correspondence Address: WYETH, PATENT LAW GROUP, FIVE GIRALDA FARMS, MADISON, NJ, 07940, US

(73) Assignee: Wyeth Holdings Corporation, Madison, NJ (US)

(21) Appl. No.: 10/740,042

(22) Filed: Dec. 18, 2003

Related U.S. Application Data
(60) Provisional application No. 60/436,039, filed on Dec. 23, 2002.

(51) Int. Cl. C11D 1/28; A61K 31/255; A61K 31/225

(52) U.S. Cl. 514/547; 514/550; 514/558; 514/517; 554/90

(57) ABSTRACT

This disclosure describes antifungal antibiotics designated Hygroscopene A, Hygroscopene B and Hygroscopene C to their production by fermentation, to methods for recovery and concentration from the crude solutions, to a process for purification and to new analogs of Hygroscopene A, Hygroscopene B and Hygroscopene C prepared by synthetic procedures.

“This application claims priority from copending provisional application, application No. 60/436,039 filed Dec. 23, 2002 the entire disclosure of which is hereby incorporated by reference.”

### FIELD OF THE INVENTION

The invention relates to new antifungal antibiotics designated Hygroscopene A, Hygroscopene B and Hygroscopene C, to their production by fermentation, to methods for recovery and concentration from the crude solutions, to a process for purification and to new analogs of Hygroscopene A, Hygroscopene B and Hygroscopene C prepared by synthetic procedures.

### BACKGROUND OF THE INVENTION

Fungi do not normally cause disease. However, under certain conditions such as with patients who have had organ transplants, chemotherapy, and those with weak immune systems, or with HIV/AIDS, fungal infections can require lengthy care under a physician. For instance, 12-15% or all hospital acquired infections are fungal. Commonly used antifungal agents include Amphotericin B and nystatin. New improved antibiotics are continually in demand as resistance to many commonly used antifungal agents increases over time.

Because fungal infections can require long hospital stays, the medical community recognizes that there is an ongoing need for additional antibiotics.

Antibiotics described in the literature include: \overline{O}MURA, S. & H. TANAKA: Production, structure, and antifunfal activity of polyene macrolides. In Macrolide Antibiotics Chemistry, Biology, and Practice. Ed., S. \overline{O}MURA, pp 351-405, Acadamic Press, Inc., New York, 1984; PANDEY, R. C.; K. L. RINEHART Jr. & D. S. MILLINGTON: The structures of Dermostatins A and B. Hidustan Antibiotics Bulletin 22:47-61,1980; MECHLINSKI, W.; C. P. SCHAFFNER, P. GANIS & G. AVITABILE: Structure and absolute configuration of the polyene macrolide antibiotic amphotericin B. Tetrahedron Lett. 1970: 3873-3876,1970; CHONG, C. N. & R. W. RICKARDS: Macrolide antibiotic studies. XVI. The structure of nystatin. Tetrahedron Lett. 1970: 5145-5148,1970; PANDEY, R. C. & K. L. RINEHART, Jr.: Polyene Antibiotics. V. Characterization of components of the filipin complex by mass spectrometry. J. Antibiotics. 23: 414-417,1970; CIFTCI T.; T. A. BORKMAN, L. E. MCDANIEL & C. P. SCHAFFNER: Comparative analysis of hexaene antibiotics. J. Antibiotics 37: 876-884,1984; KARADZIC, I. M.; G. D. GOJGIC & J. I. VUCETIC: Hexaene H-85, a hexaene macrolide complex. J. Antibiotics 44: 1452-1453, 1991. The structure of Linearmycin A is described in S. SAKUDA, et al, Tetrahedron Letters, 36 (16), 2777-2780 (1995).

Streptomyces hygroscopicus ATCC 29253 was originally described as a rapamycin producing strain (VEZINA, C., A. KUDLESKI, & S. N. SEGHAL: Rapamycin (AY-22989), a new antifungal antibiotic. J. Antibiotics 28:721-726, 1975). This strain is also reported to produce other secondary metabolites, namely elaiophylin and nigericin (FANG, A. G. K. WONG, A. L. DEMAIN. J. Antibiotics 53: 158-162, 2000). Nigericin is reported to exhibit antifungal activity while elaiophylin is reported to have antibacterial activity. It is well known in the art that growth conditions and the composition of the medium can have a profound influence on the spectrum of compounds produced by an organism. When grown under appropriate conditions, S. hygroscopicus ATCC 25293 produces unique antifungal compounds that are distinct from any compounds previously known to be produced by this strain. This invention describes these novel antifungal compounds, designated hygroscopenes, that are different from the disclosed antibiotics referenced above. Hygroscopenes are novel linear polyenes containing a conjugated hexaene structure. The invention further provides a process for the production and isolation of Hygroscopenes.

### BRIEF SUMMARY OF THE INVENTION

The invention relates to antibiotics Hygroscopene A, Hygroscopene B and Hygroscopene C, the production of these antibiotics by fermentation, to methods for the recovery and concentration of these antibiotics from crude solution, and to processes for the purification of these antibiotics.

The invention includes within its scope the antibiotics in diluted form, as crude concentrates and in pure form. The novel antibiotics are useful as antifungal agents.

The new antibiotics designated Hygroscopenes are formed during the cultivation under controlled conditions of the microorganism of Streptomyces hygroscopicus ATCC29253.

The invention further includes within its scope new synthetically modified compounds of Hygroscopene A, Hygroscopene B and Hygroscopene C, and further includes acyl derivatives and methyl esters.

This invention is concerned with compounds of Formula I:

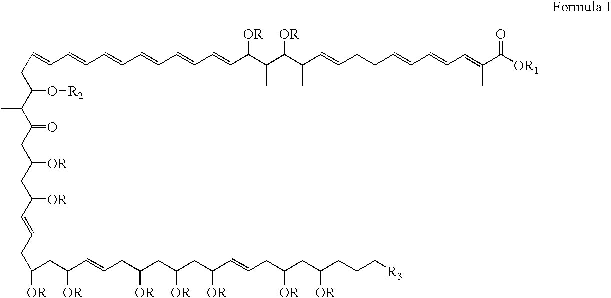

wherein:

R is H or —COCH₃;

R₁ is H or lower alkyl of 1 to 4 carbon atoms;

R₂ is H or —SO₃H;

R₃ is

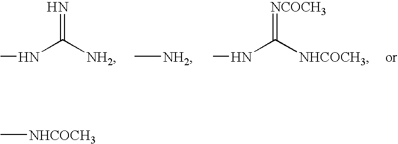

and pharmaceutically acceptable salts thereof.

Preferred compounds of Formula I include:

a) Hygroscopene A Methyl Ester;

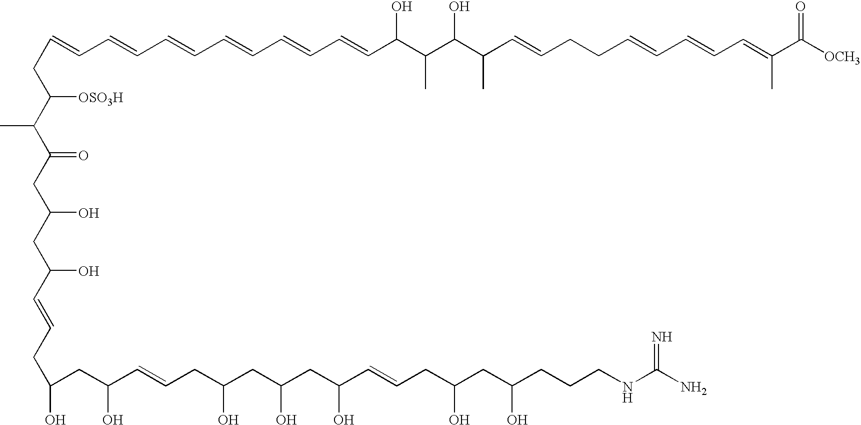

b) Hygroscopene B Methyl Ester;

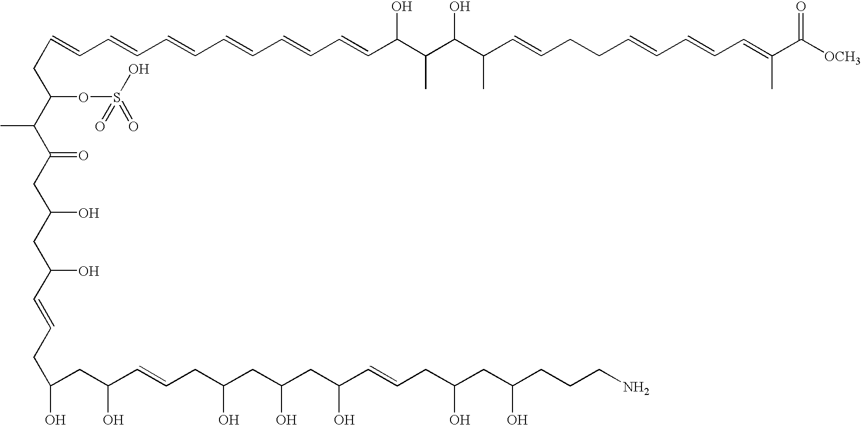

c) Acetyl-hygroscopene A and

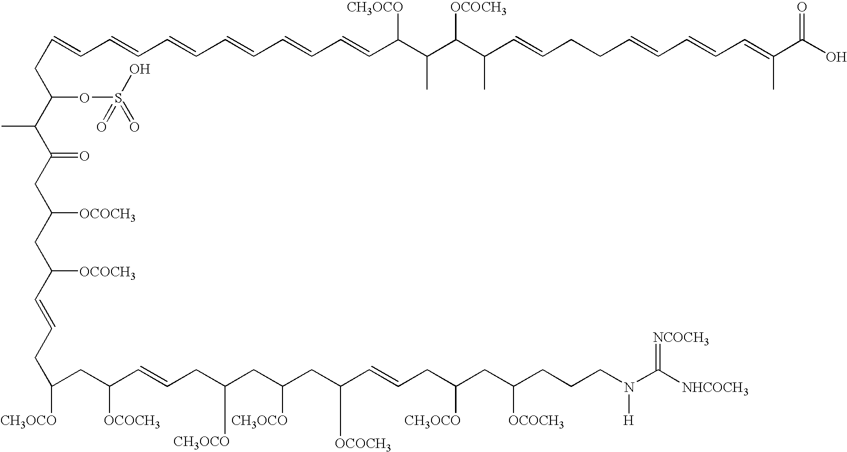

d) Acetyl-hygroscopene B.

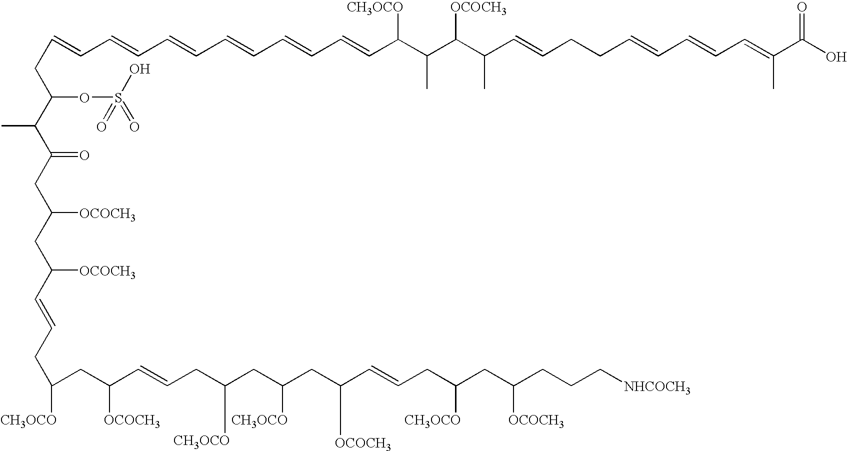

In particular, the invention is concerned with Hygroscopene A, Hygroscopene B and Hygroscopene C.

The structure of antibiotic Hygroscopene A is:

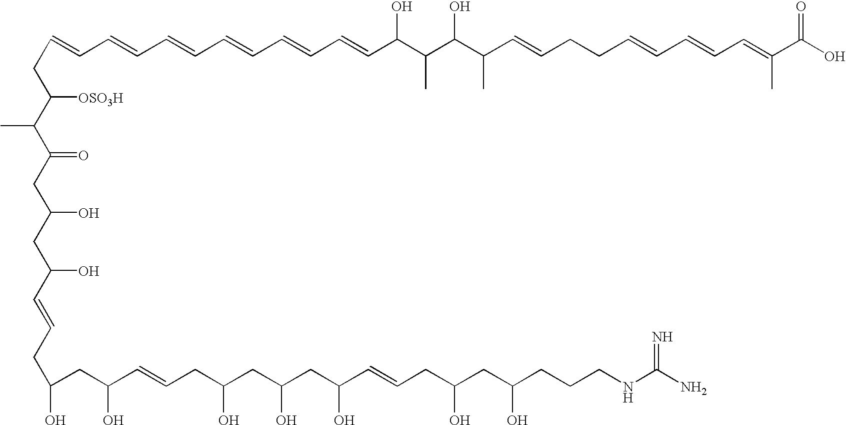

The structure of antibiotic Hygroscopene B is:

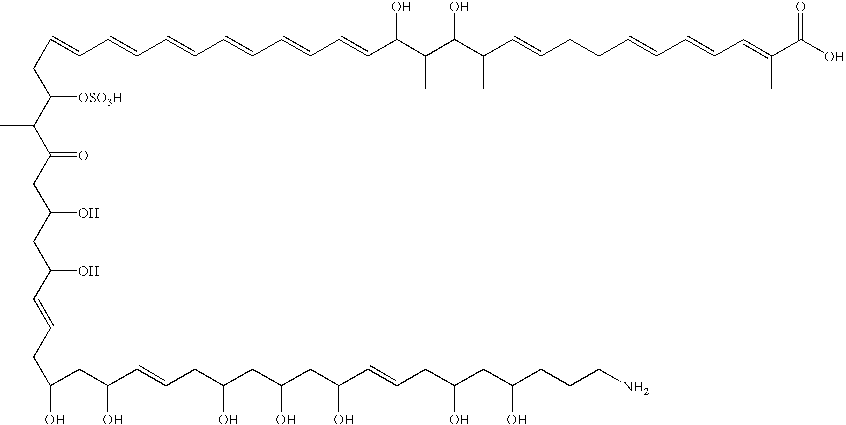

The structure of the antibiotic Hygroscopene C is:

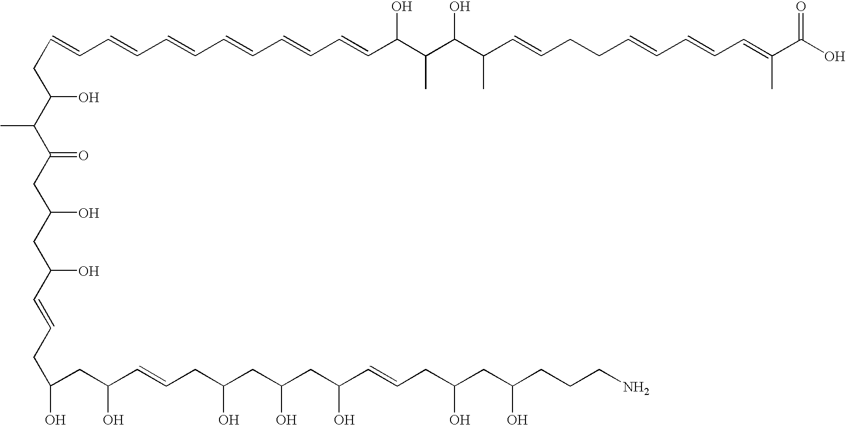

### BRIEF DESCRIPTION OF THE DRAWINGS

FIG. 1. Characteristic UV-VIS spectrum of Hygroscopene A (in CH₃OH)

FIG. 2. Characteristic Mass Spectrum of Hygroscopene A (Negative ESI-MS)

FIG. 3. Characteristic proton nuclear magnetic resonance (NMR) spectrum of Hygroscopene A (400 MHz, CD₃OD).

FIG. 4. Characteristic carbon-13 nuclear magnetic resonance spectrum (NMR) spectrum of Hygroscopene A (400 MHz, CD₃OD).

FIG. 5. Characteristic UV-VIS spectrum of Hygroscopene B (in CH₃OH)

FIG. 6. Characteristic Mass Spectrum of Hygroscopene B (Negative ESI-MS)

FIG. 7. Characteristic proton nuclear magnetic resonance (NMR) spectrum of Hygroscopene B (400 MHz, CD₃OD).

FIG. 8. Characteristic carbon-13 nuclear magnetic resonance spectrum (NMR) spectrum of Hygroscopene B (400 MHz, CD₃OD).

FIG. 9. Characteristic UV-VIS spectrum of Hygroscopene C (in CH₃OH)

FIG. 10. Characteristic Mass spectrum of Hygroscopene C (Negative ESI-MS)

FIG. 11. Characteristic proton nuclear magnetic resonance (NMR) spectrum of Hygroscopene C (400 MHz, CD₃OD).

FIG. 12. Characteristic carbon-13 nuclear magnetic resonance spectrum (NMR) spectrum of Hygroscopene C (400 MHz, CD₃OD).

### DESCRIPTION OF THE PREFERRED EMBODIMENTS

The invention relates to new Hygroscopene antibiotics, to the production of the antibiotics by fermentation, to methods for the recovery and concentration of the antibiotics from crude solutions, and to processes for the purification of the antibiotics. The invention includes within its scope the new antibiotics in diluted form, as crude concentrate and in pure form. The novel antibiotics are useful as antifungal agents. The new antibiotics Hygroscopene A, Hygroscopene B and Hygroscopene C are formed during the fermentation of Streptomyces hygroscopicus ATCC29253.

The physiochemical characteristics for Hygroscopene A, Hygroscopene B and Hygroscopene C are described below:

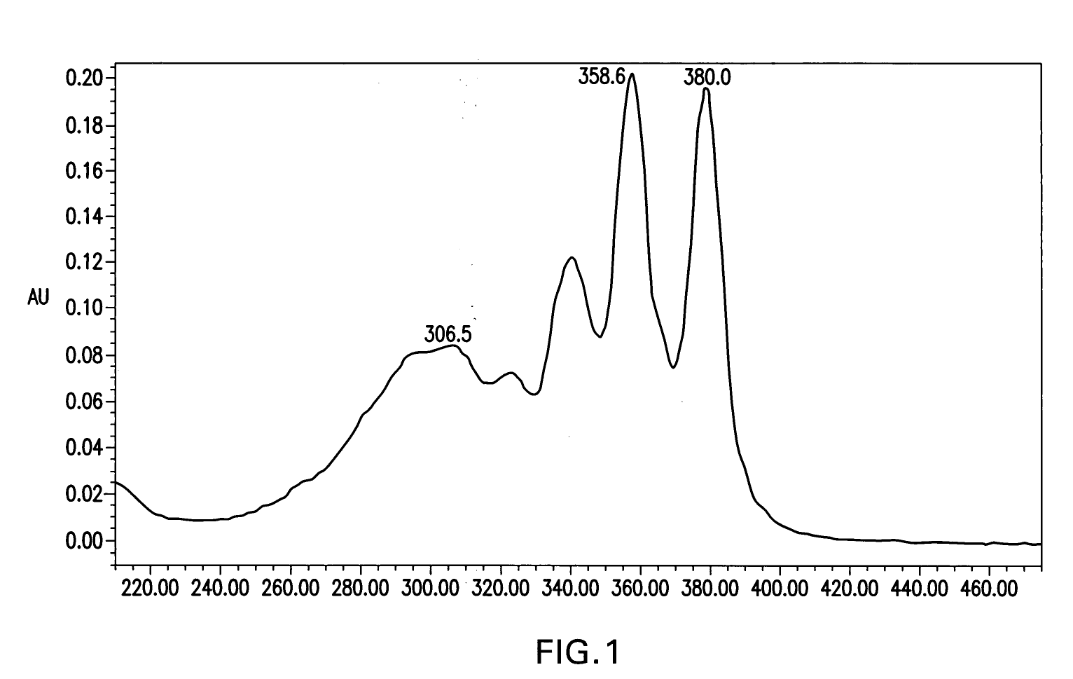

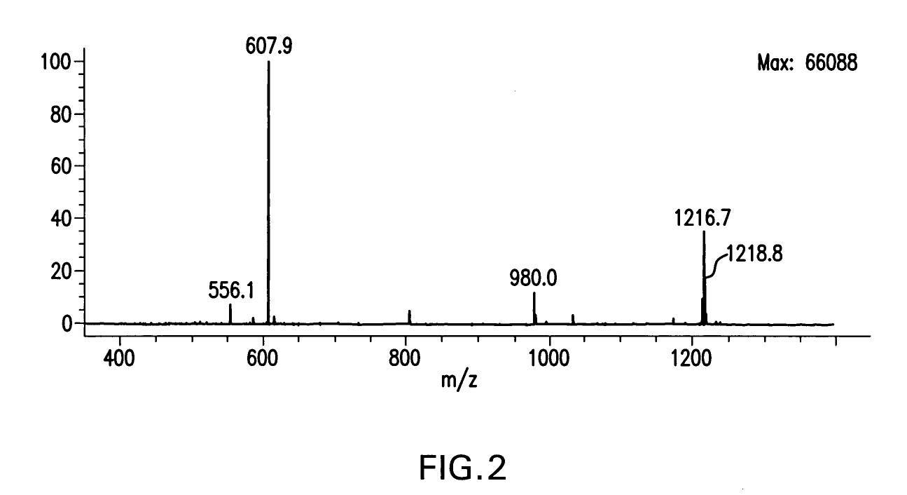

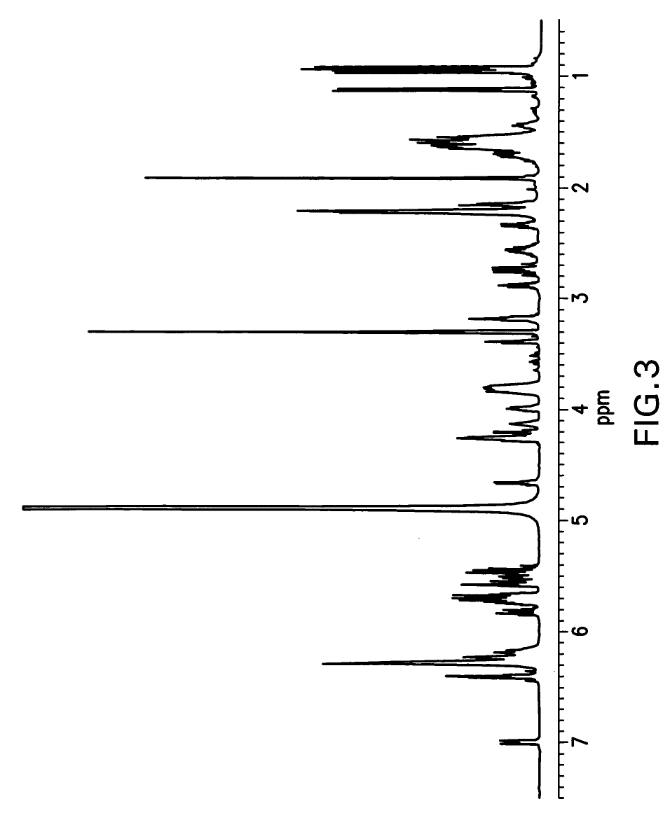

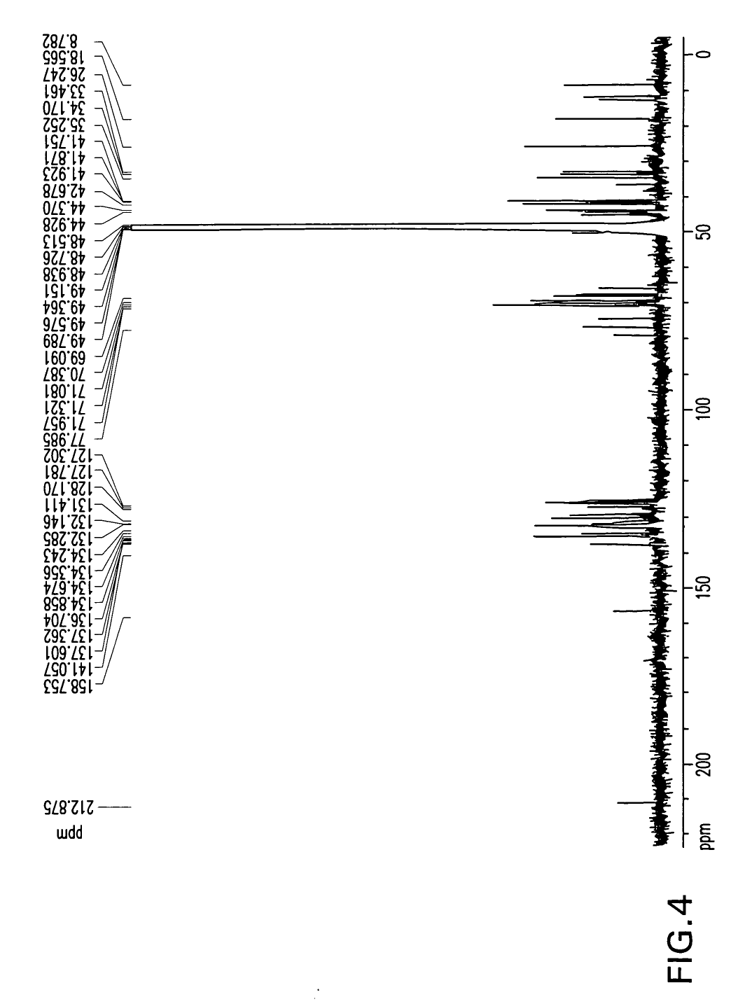

Description
The Physico-chemical Properties of Hygroscopene A:

| | |
|---|---|
| Appearance | Yellow amphoteric powder |
| Optical rotation [α]²⁵ _{D} | −16.5° (c 0.097, MeOH) |
| UV λₘₐₓ (CH₃OH) (FIG. 1) | 295 (sh), 306, 322, 341, 359, 380 nm |
| IR νₘₐₓ (KBr) | 3421. 3015, 2925, 2855, 1669, 1632, 1240, 972 |
| Apparent Molecular Formula | C₆₃H₉₉N₃O₁₈S |
| Molecular weight | 1217 |
| Negative ESI-MS (FIG. 2) | 1216.7 [M − H]⁻ |
| HRMS (negative) | 1216.65684 (cald. 1216.65714 for C₆₃H₉₈N₃O₁₈S) |
| Proton nuclear magnetic | 7.00 (1H, bd, H-3), 6.41 (1H, H-4), 6.39 (1H, H-5), |
| resonance spectrum: as | 6.28-6.17 (o, 11H, H-6, H-17, H-18, H-19, H-20, H-21, |
| shown in FIG. 3. | H-22, H-23, H-24, H-25, H-26), 5.81 (1H, dt, H-7), |
| (400 MHz, CD₃OD) | 5.72-5.45 (o, 9H, m, H-10, H-16, H-27, H-36, H-37, H- |
| | 42, H-43, H-50, H-51), 5.43 (1H, dd, H-11), 4.66 (1H, |
| | dt, H-29), 4.27-4.24 (3H, m, H-41. H-49, H-15), 4.20 |
| | (1H, dt, H-35), 4.13 (1H, m, H-33), 3.99 (1H, m, H-47), |
| | 3.83-3.77 (o, 4H, H-39, H-45, H-53, H-55), 3.38 (1H, t, |
| | H-13), 3.18 (2H, m, H-58), 2.88 (1H, q, H-30), 2.76 |
| | (1H, dd, H-32), 2.71 (1H, dd, H-32′), 2.56 (2H, m, H- |
| | 28), 2.33 (1H, m, H-12), 2.21 (8H, o, H-8, H-38, H-44, |
| | H-52), 2.14 (2H, dt, H-9), 1.91 (3H, s, H-60), 1.72-1.52 |
| | (13H, m, H-34, H-40, H-46, H-48, H-54, H-57, H-14), |
| | 1.43 (2H, m, H-56) 1.12 (3H, d, H-63), 0.96 (3H, d, H- |
| | 61), 0.93 (3H, d, H-62), 17 exchangeable protons |
| Carbon-13 nuclear magnetic | 177.0 (C-1), 133.8 (C-2), 136.0 (C-3), 128.3 (C-4), |
| resonance spectrum: as | 138.3 (C-5), 132.6 (C-6), 137.6 (C-7), 34.2 (C-8), 33.6 |
| shown in FIG. 4 | (C-9), 131.5 (C-10), 134.7 (C-11), 41.8 (C-12), 77.9 |
| (400 MHz, CD₃OD) | (C-13), 42.5 (C-14), 75.4 (C-15), 137.3 (C-16), 132.1 |
| | (C-17), 134.2 (C-18), 134.3 (C-19), 133.9 (C-20), |
| | 134.3 (C-21), 134.4 (C-22), 134.6 (C-23), 134.7 (C- |
| | 24), 133.5 (C-25), 135.2 (C-26), 130.9 (C-27), 37.2 (C- |
| | 28), 80.1 (C-29), 51.2 (C-30), 212.9 (C-31), 51.0 (C- |
| | 32), 66.8 (C-33), 45.2 (C-34), 72.0 (C-35), 136.7 (C- |
| | 36), 129.3 (C-37), 41.9 (C-38), 69.1 (C-39), 45.0 (C- |
| | 40), 70.4 (C-41), 137.5 (C-42), 127.8 (C-43), 41.7 (C- |
| | 44), 71.3 (C-45), 44.8 (C-46), 68.5 (C-47), 45.8 (C-48), |
| | 70.4 (C-49), 137.4 (C-50), 128.2 (C-51), 41.7 (C-52), |
| | 71.3 (C-53), 44.4 (C-54), 71.0 (C-55), 35.3 (C-56), |
| | 26.2 (C-57), 42.6 (C-58), 158.8 (C-59), 14.2 (C-60), |
| | 18.6 (C-61), 8.9 (C-62), 12.0 (C-63) |
| Solubility | Soluble in MeOH, EtOH, DMSO |

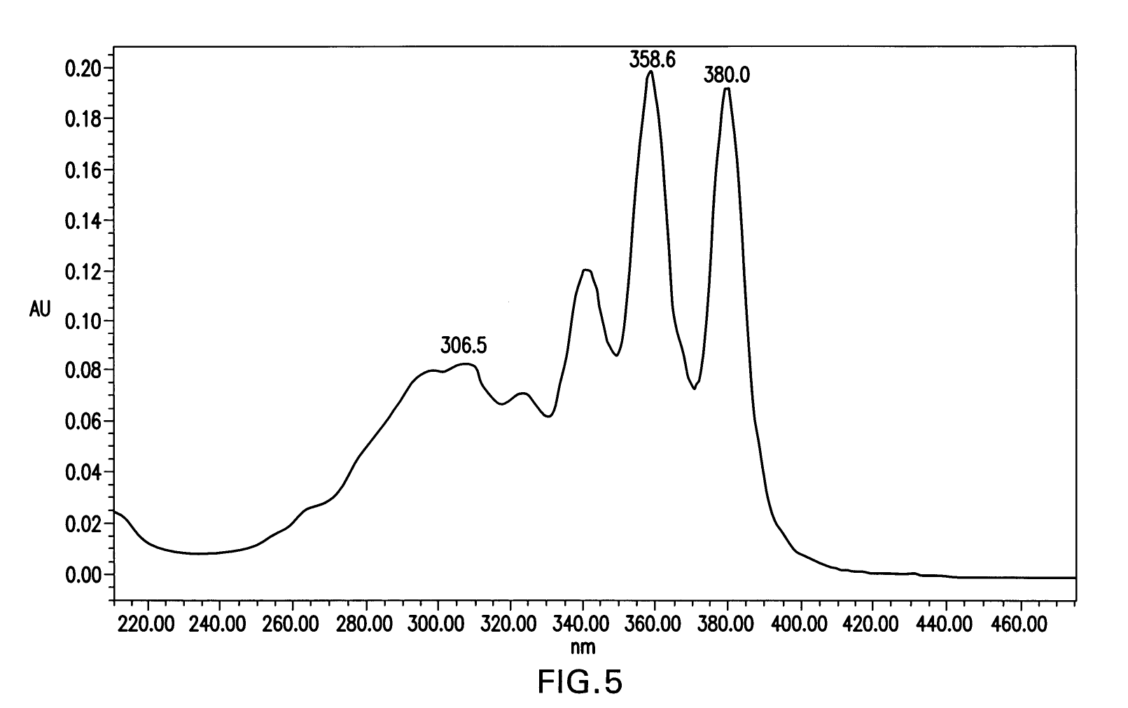

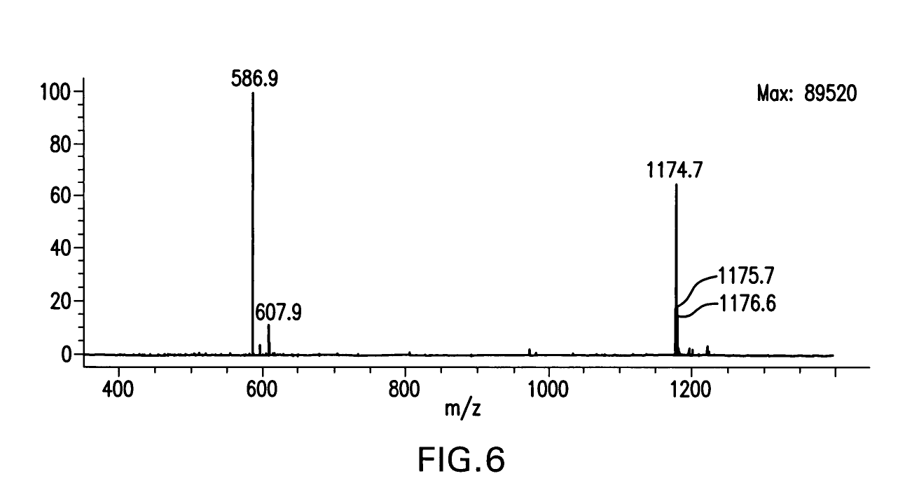

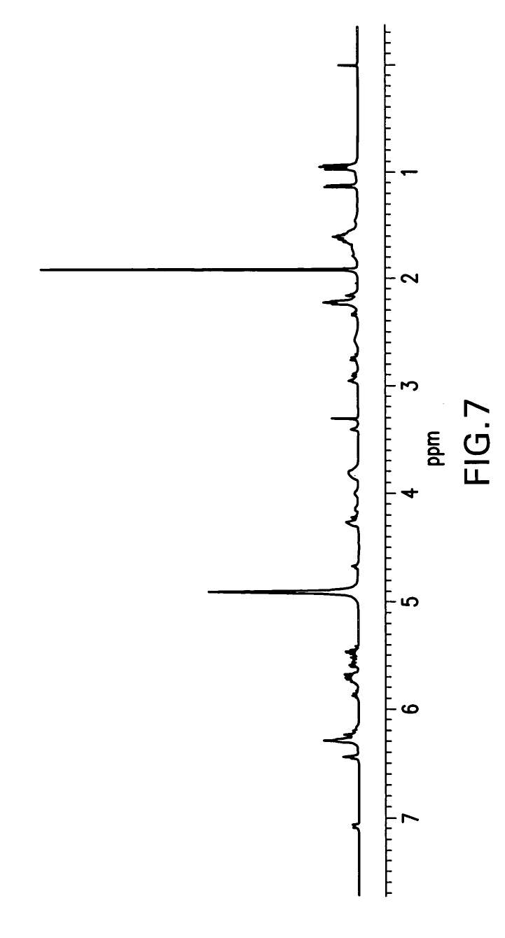

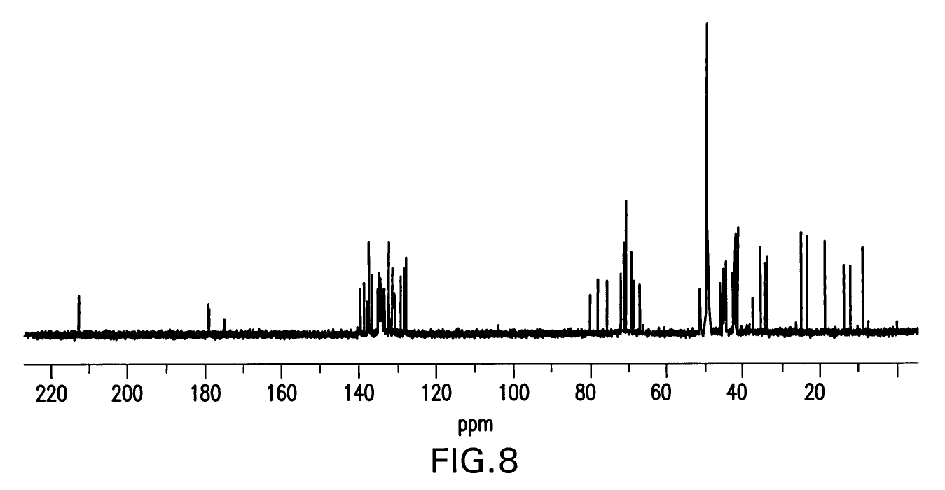

The Physico-chemical Properties of Hygroscopene B are:

| | |
|---|---|
| Appearance | Yellow amphoteric powder |
| UV λₘₐₓ (CH₃OH) (FIG. 5) | 295 (sh), 306, 322, 341, 359, 380 nm |
| IR νₘₐₓ (KBr) | 3419. 3015, 2925, 2855, 1669, 1632, 1240, 972 |
| Molecular Formula | C₆₂H₉₇NO₁₈S |
| Molecular weight | 1175 |
| Negative ESI-MS (FIG. 6) | 1174.6 [M − H]⁻ |
| HRMS (positive) | 1176.64787 (cald. 1176.64991 for C₆₂H₉₈NO₁₈S) |
| Proton nuclear magnetic | 7.05 (1H, bd, H-3), 6.44 (1H, H-4), 6.42 (1H, H-5), 6.25-6.21 |
| resonance spectrum (as | (9H, o, H-17, H-18, H-19, H-20, H-21, H-22, H-23, |
| shown in FIG. 7) (400 MHz, | H-24, H-25) 6.19 (1H, m H-6), 6.17 (1H, m, H-26), 5.87 |
| CD₃OD) | (1H, dt, H-7), 5.72-5.66 (5H, o, H-16, H-27, H-37, H-43, |
| | H-51), 5.59-5.46 (4H, m, H-10, H-36, H-43, H-50), 5.44 |
| | (1H, dd, H-11), 4.67 (1H, dt, H-29), 4.29-4.22 (3H, m, |
| | H-41. H-49, H-15), 4.21 (1H, dt, H-35), 4.14 (1H, m, H- |
| | 33), 3.99 (1H, m, H-47), 3.86-3.79 (o, 4H, H-39, H-45, |
| | H-53, H-55), 3.41 (1H, dd, H-13), 2.95 (2H, m, H-58), |
| | 2.88 (1H, q, H-30), 2.76 (1H, dd, H-32), 2.73 (1H, dd, H- |
| | 32′), 2.55 (2H, m, H-28), 2.33 (1H, m, H-12), 2.23-2.21 |
| | (8H, o, H-8, H-38, H-44, H-52), 2.14 (2H, dt, H-9), 1.93 |
| | (3H, s, H-59), 1.78 (2H, m, H57), 1.73-1.58 (13H, m, H- |
| | 34, H-40, H-46, H-48, H-54, H-14), 1.43 (2H, m, H-56), |
| | 1.12 (3H, d, H-62), 0.95 (3H, d, H-60), 0.91 (3H, d, H- |
| | 61), 15 exchangeable protons |
| Carbon-13 nuclear magnetic | 178.7 (C-1), 130.6 (C-2), 139.6 (C-3), 128.0 (C-4), |
| resonance spectrum (as | 138.6 (C-5), 132.2 (C-6), 137.9 (C-7), 34.0 (C-8), 33.4 |
| shown in FIG. 8) | (C-9), 131.3 (C-10), 134.7 (C-11), 41.7 (C-12), 77.9 (C- |
| (400 MHz, CD₃OD) | 13), 42.3 (C-14), 75.4 (C-15), 137.1 (C-16), 132.0 (C- |
| | 17), 134.1 (C-18), 134.3 (C-19), 133.8 (C-20), 134.2 (C- |
| | 21), 134.4 (C-22), 134.5 (C-23), 134.6 (C-24), 133.3 (C- |
| | 25), 135.0 (C-26), 130.8 (C-27), 37.0 (C-28), 79.9 (C- |
| | 29), 51.0 (C-30), 212.8 (C-31), 50.8 (C-32), 66.7 (C-33), |
| | 45.0 (C-34), 71.8 (C-35), 136.5 (C-36), 129.1 (C-37), |
| | 41.8 (C-38), 68.9 (C-39), 44.7 (C-40), 70.2 (C-41), |
| | 137.4 (C-42), 127.6 (C-43), 41.6 (C-44), 71.2 (C-45), |
| | 44.7 (C-46), 68.4 (C-47), 45.7 (C-48), 70.2 (C-49), 137.1 |
| | (C-50), 127.7 (C-51), 41.6 (C-52), 71.1 (C-53), 44.2 (C- |
| | 54), 70.7 (C-55), 35.0 (C-56), 24.9 (C-57), 40.9 (C-58), |
| | 13.5 (C-59), 18.4 (C-60), 8.6 (C-61), 11.8 (C-62) |
| Solubility | Soluble in MeOH, EtOH, DMSO |

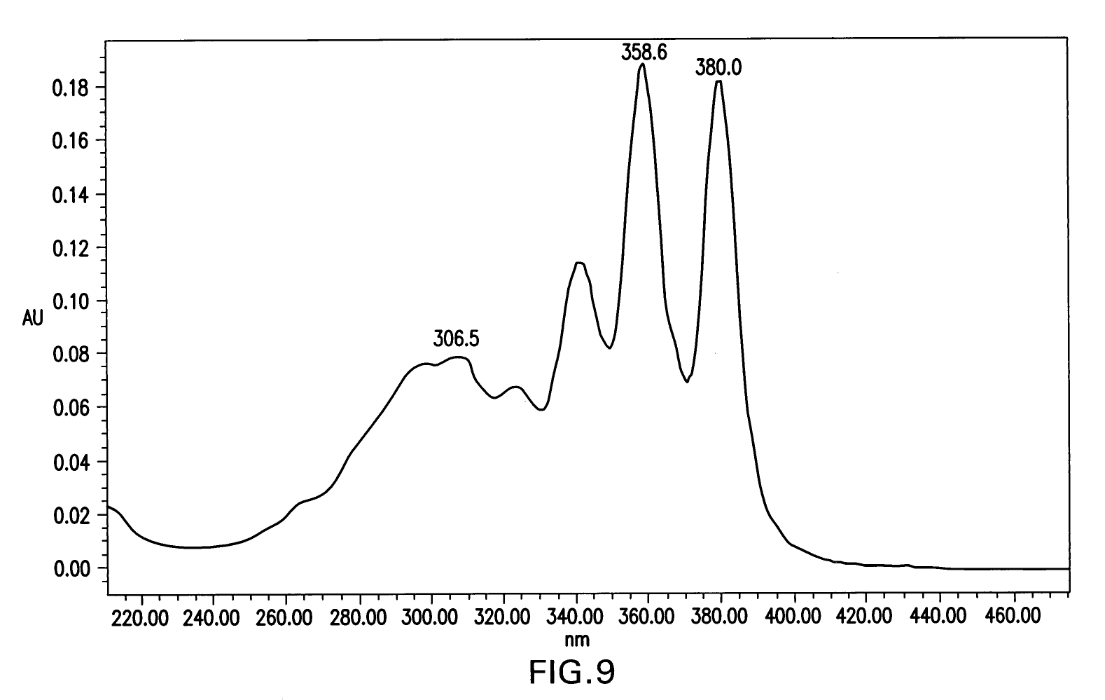

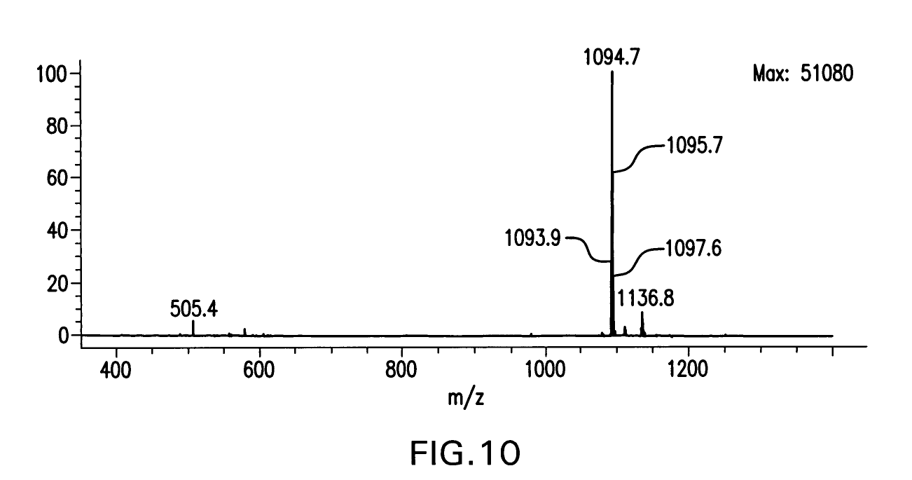

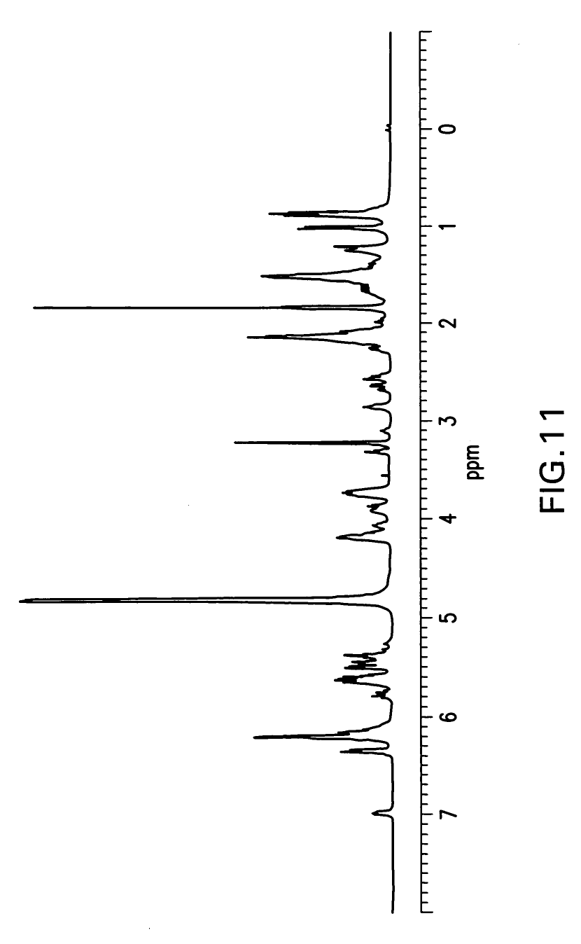

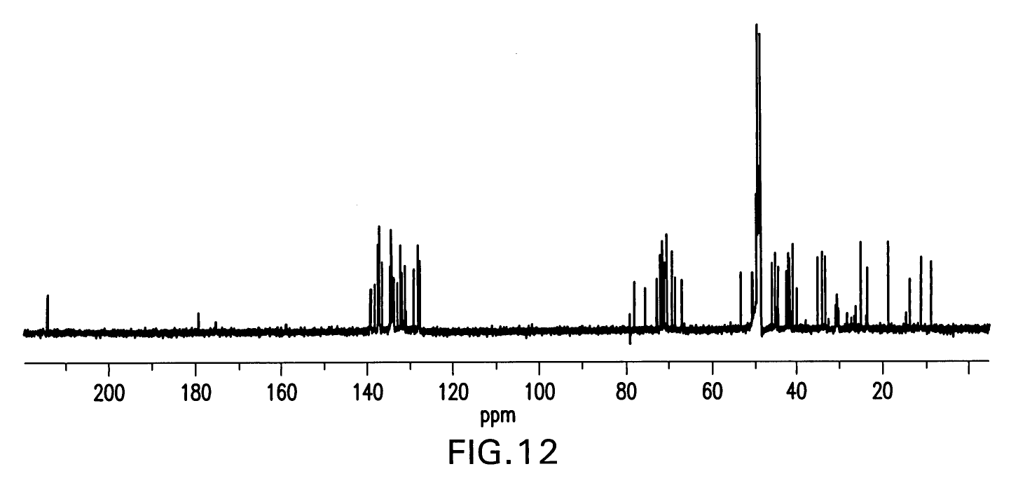

The Physico-chemical Properties of Hygroscopene C are:

| | |
|---|---|
| Appearance | Yellow amphoteric powder |
| UV λₘₐₓ (CH₃OH) (FIG. 9) | 295 (sh), 306, 322, 341, 359, 380 nm |
| IR νₘₐₓ (KBr) | 3419. 3015, 2925, 2855, 1669, 1632, 1240, 972 |
| Molecular Formula | C₆₂H₉₇NO₁₅ |
| Molecular weight | 1095 |
| Negative ESI-MS (FIG. 10) | 1094.6 [M − H]⁻ |
| HRMS (positive) | 1096.69213 (cald. 1096.69310 for C₆₂H₉₈NO₁₅) |
| Proton nuclear magnetic | 6.99 (1H, bd, H-3), 6.35 (1H, H-4), 6.32 (1H, H-5), |
| resonance spectrum (as | 6.20-6.15 (9H, o, H-17, H-18, H-19, H-20, H-21, H-22, |
| shown in FIG. 11) | H-23, H-24, H-25) 6.15 (1H, m H-6), 6.12 (1H, m, H- |
| (400 MHz, CD₃OD) | 26), 5.79 (1H, dt, H-7), 5.64-5.60 (5H, o, H-16, H-27, |
| | H-37, H-43, H-51), 5.59-5.50 (4H, m, H-10, H-36, H- |
| | 43, H-50), 5.45 (1H, dd, H-11), 4.20-4.18 (3H, m, H- |
| | 41, H-49, H-15), 4.17 (1H, dt, H-35), 4.14 (1H, m, H- |
| | 33), 3.92-3.87 (2H, m, H-47, H-29), 3.74-3.71 (o, 4H, |
| | H-39, H-45, H-53, H-55), 3.33 (1H, dd, H-13), 3.08 |
| | (1H, m, H-30), 2.87 (2H, m, H-58), 2.66 (1H, dd, H-32), |
| | 2.63 (1H, dd, H-32′), 2.54 (2H, m, H-28), 2.27 (1H, m, |
| | H-12), 2.22-2.11 (8H, o, H-8, H-38, H-44, H-52), 2.07 |
| | (2H, dt, H-9), 1.83 (3H, s, H-59), 1.65 (2H, m, H57), |
| | 1.73-1.58 (13H, m, H-34, H-40, H-46, H-48, H-54, H- |
| | 14), 1.24 (2H, m, H-56), 1.02 (3H, d, H-62), 0.88 (3H, |
| | d, H-60), 0.84 (3H, d, H-61) |
| Carbon-13 nuclear magnetic | 179.1 (C-1), 130.9 (C-2), 139.1 (C-3), 128.0 (C-4), |
| resonance spectrum (as | 138.2 (C-5), 132.2 (C-6), 137.4 (C-7), 34.0 (C-8), 33.4 |
| shown in FIG. 12) | (C-9), 131.9 (C-10), 134.5 (C-11), 41.8 (C-12), 77.8 |
| (400 MHz, CD₃OD) | (C-13), 42.4 (C-14), 75.3 (C-15), 137.1 (C-16), 132.0 |
| | (C-17), 134.0 (C-18), 134.2 (C-19), 133.7 (C-20), |
| | 134.1 (C-21), 134.3 (C-22), 134.4 (C-23), 134.4 (C- |
| | 24), 133.0 (C-25), 134.6 (C-26), 131.3 (C-27), 39.8 (C- |
| | 28), 72.6 (C-29), 53.1 (C-30), 212.8 (C-31), 50.4 (C- |
| | 32), 66.7 (C-33), 45.0 (C-34), 71.8 (C-35), 136.5 (C- |
| | 36), 129.1 (C-37), 42.3 (C-38), 68.9 (C-39), 44.9 (C- |
| | 40), 70.1 (C-41), 137.4 (C-42), 127.6 (C-43), 41.5 (C- |
| | 44), 71.1 (C-45), 44.7 (C-46), 68.3 (C-47), 45.7 (C-48), |
| | 70.2 (C-49), 137.2 (C-50), 127.7 (C-51), 41.6 (C-52), |
| | 71.0 (C-53), 44.2 (C-54), 70.7 (C-55), 35.0 (C-56), |
| | 24.9 (C-57), 40.8 (C-58), 13.6 (C-59), 18.4 (C-60), 8.6 |
| | (C-61), 11.1 (C-62) |
| Solubility | Soluble in MeOH, EtOH, DMSO |

Additionally, compounds of this invention may be prepared according to Scheme I.

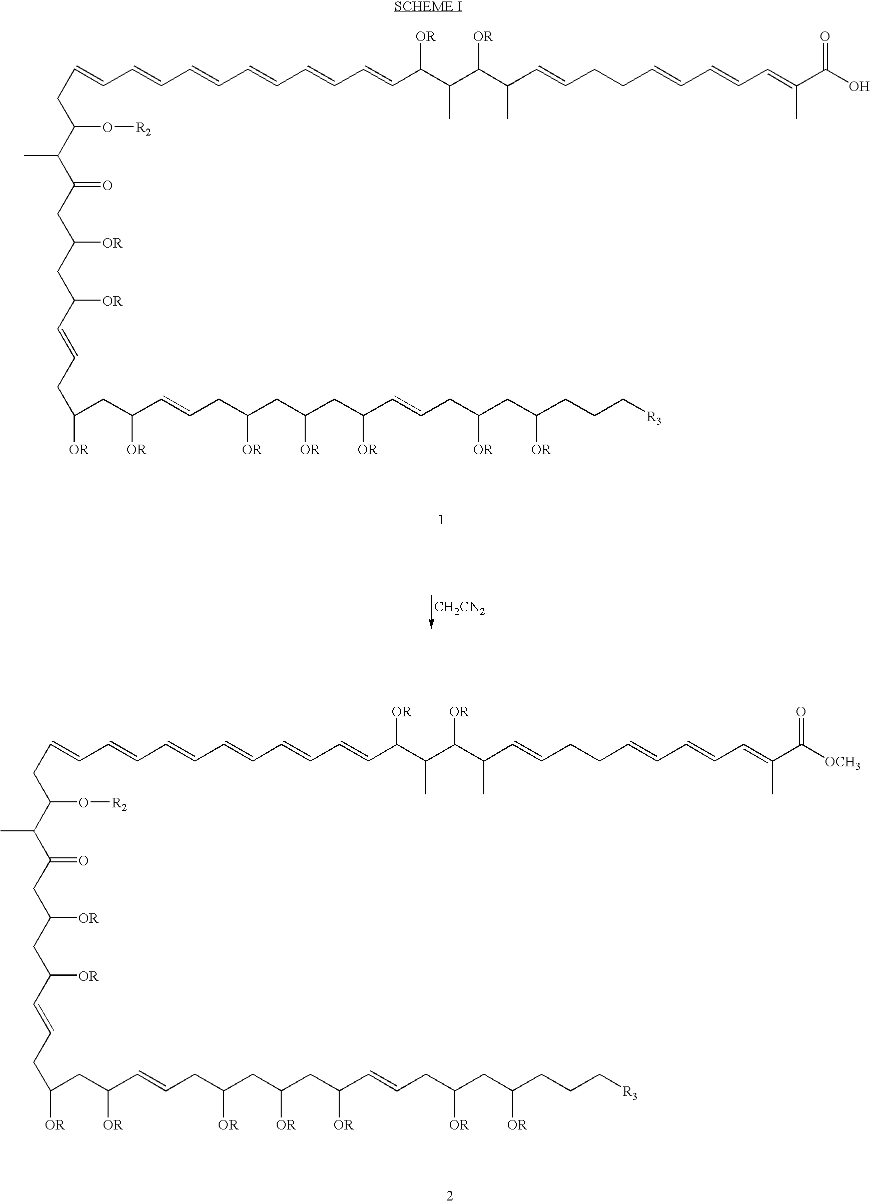

As described in Scheme I, carboxylic acid 1 where R, R₂ and R₃ are hereinbefore defined are reacted with diazomethane in methanol at room temperature to afford ester 2.

As shown in Scheme II, carboxylic acid 3 where R₃ is

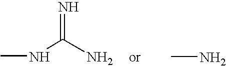

is acylated with acetic anhydride in pyridine in the presence of dimethylaminopyridine (DMAP) to afford acyl derivative 4 where R₃ is

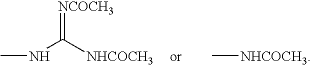

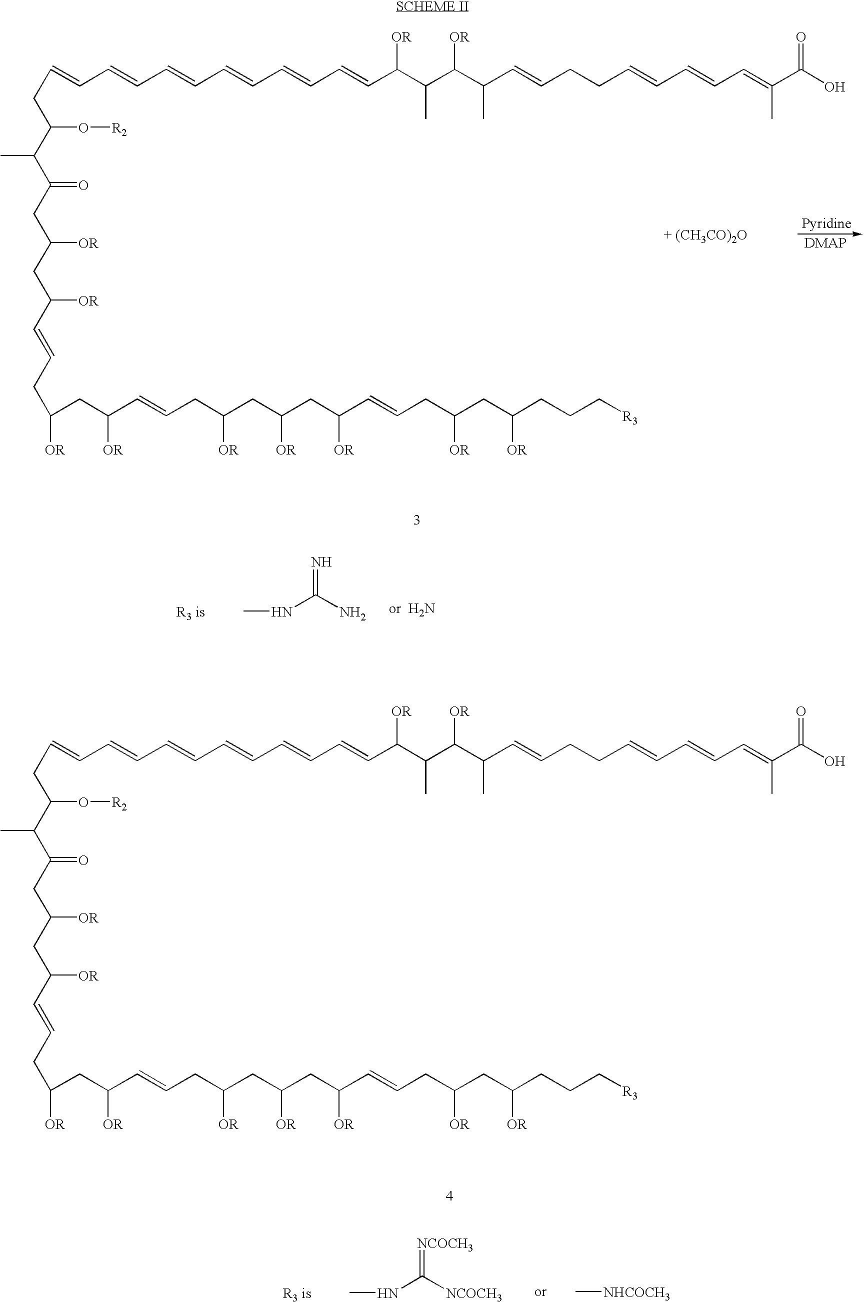

The new antifungal agents Hygroscopene A, Hygroscopene B, and Hygroscopene C are formed during the cultivation under controlled conditions of Streptomyces hygroscopicus ATCC29253. However, the production of Hygroscopene A and Hygroscopene B and Hygroscopene C is not limited to this particular organism. In fact, it is desired and intended to include the use of naturally-occurring mutants of this organism, as well as induced mutants produced from this organism by various classical mutagenic means known to those skilled in the art. It is also desired and intended to include inter- and intraspecific genetic recombinants produced by genetic techniques know to those skilled in the art such as for example, conjugation, transduction and genetic engineering techniques.

It is a further object of the invention to provide a method of treating fungal infections, in mammals, especially man, in need thereof with an effective amount of compounds of Formula I and pharmaceutically acceptable salts thereof.

It is an additional object of the invention to provide a pharmaceutical composition of compounds of Formula I and pharmaceutically acceptable salts thereof in the presence of one or more pharmaceutically acceptable carriers.

It is a further object of the invention to provide a method of treating fungal infections, in mammals, especially man, in need thereof with an effective amount of compounds Hygroscopene A, Hygroscopene B or Hygroscopene C and pharmaceutically acceptable salts thereof.

It is an additional object of the invention to provide a pharmaceutical composition of compounds Hygroscopene A, Hygroscopene B or Hygroscopene C and pharmaceutically acceptable salts thereof in the presence of one or more pharmaceutically acceptable carriers.

Pharmaceutically acceptable salts are formed from organic and inorganic acids, for example, acetic, propionic, lactic, citric, tartaric, succinic, fumaric, maleic, malonic, mandelic, malic, phthalic, hydrochloric, hydrobromic, phosphoric, nitric, sulfuric, methanesulfonic, naphthalenesulfonic, benzenesulfonic, toluenesulfonic, camphorsulfonic, and similarly known acceptable acids when a compound of this invention contains a basic moiety. Pharmaceutically acceptable salts may also be formed from organic and inorganic bases, preferably alkali metal salts, for example, sodium, lithium, or potassium, when a compound of this invention contains an acidic moiety.

The compounds of this invention may contain an asymmetric carbon atom and some of the compounds of this invention may contain one or more asymmetric centers and may thus give rise to optical isomers and diastereomers. While shown without respect to stereochemistry, the present invention includes such optical isomers and diastereomers; as well as the racemic and resolved, enantiomerically pure R and S stereoisomers; as well as other mixtures of the R and S stereoisomers and pharmaceutically acceptable salts thereof. It is recognized that one optical isomer, including diastereomer and enantiomer, or stereoisomer may have favorable properties over the other. Thus when disclosing and claiming the invention, when one racemic mixture is disclosed, it is clearly contemplated that both optical isomers, including diastereomers and enantiomers, or stereoisomers substantially free of the other are disclosed and claimed as well.

Biological Activity

Standard Pharmacological Test Procedures

The antifungal activity of Hygroscopene A is determined by a broth microdilution method as recommended by the National Committee for Clinical Laboratory Standard (NCCLS) (1, 2). Sterile, disposable, multiwell plates (96 U-shaped wells) containing serial dilutions of Hygroscopene A and a control drug (Amphotericin B) are prepared in a 100 μl volume with RPMI-1640 broth supplemented with 0.2% sodium bicarbonate and 0.165 MOPS [3-(N-morpholino) propanesulfonic acid] buffer. RPMI-1640 is a completely synthetic medium that supports the growth of most fungi. Antimicrobial agent concentrations ranged from 0.06-64 μg/ml. Five colonies from 24-hour old yeast culture or 48-hour old filamentous fungi culture were suspended in 0.85% saline. The suspensions are diluted in RPMI-1640 broth and inoculated in to plates (100 μl/well) containing antimicrobial agents. The final inocula of yeast or fungi are 0.5-2.5×10³ CFU/ml and 0.4×10⁴-5×10⁴ CFU/ml respectively. Plates are incubated at 35° C. for 24-72 hours in ambient air. Hygroscopene A is tested against a panel of pathogenic yeast and filamentous fungi, which consisted of Candida sp. and Aspergillus sp. The yeast and fungal strains tested are clinical isolates or were purchased from American Type Culture Collection (ATCC) (Rockville, Md.). The minimal inhibitory concentration (MIC) is the lowest concentration of an antimicrobial agent that completely inhibited growth of the organism as detected by the unaided eye. The MIC values for yeast are determined after 24-48 hours of incubation. For filamentous fungi, the MIC is determined after 72 hours of incubation. The MIC results of representative examples of the invention are given in Table 2.

TABLE 2
Antifungal Activity of Hygroscopene A

| | | MIC (μg/ml) | | |
|---|---|---|---|---|
| | | Amphotericin- | Hygro- | Growth |
| Yeast/Fungi | I. D # | B | scopene A | Control |
| Candida albicans | 1063 | 0.125 | 2.0 | + |
| Candida albicans | 1117 | 0.125 | 1.0 | + |
| Candida albicans | ATCC | 0.25 | 2.0 | + |
| 90028 | | | | |
| Candida parapsilosis | 94-8 | 0.25 | 2.0 | + |
| Candida parapsilosis | 94-9 | 0.25 | 2.0 | + |
| Candida parapsilosis | ATCC | 0.25 | 2.0 | + |
| 90018 | | | | |
| Candida tropicalis | 94-13 | 0.25 | 1.0 | + |
| Candida tropicalis | 94-14 | 0.25 | 1.0 | + |
| Candida | ATCC | 0.25 | 2.0 | + |
| pseudotropicalis 28838 | | | | |
| Candida krussii | 94-2 | 0.50 | 1.0 | + |
| Candida lustitaniae | 94-3 | 0.125 | 1.0 | + |
| Candida rugosa | 94-10 | 0.50 | 1.0 | + |
| Aspergillus fumigatus | S-430 | 0.25 | 2.0 | + |
| Aspergillus niger | ATCC | 0.50 | 8.0 | + |
| 26933 | | | | |

### REFERENCES

1. National Committee for Clinical Laboratory Standards. 2002. Methods for Broth Dilution Antifungal Susceptibility Testing of Yeast; Approved Standard-Second Edition. M27-A2; Vol.22 No. 15. National Committee for Clinical Laboratory Standards, Wayne, Pa.

2. National Committee for Clinical Laboratory Standards. 2002. Methods for Broth Dilution Antifungal Susceptibility Testing of Filamentous Fungi; Approved Standard. M38-A; Vol. 22, No. 16. National Committee for Clinical Laboratory Standards, Wayne, Pa.

The in vitro antimicrobial results show that the compounds according to the invention has significant activity against fungal and yeast strains tested.

Antibiotics of Formula I and Hygroscopene A, Hygroscopene B and Hygroscopene C derive their utility from their antifungal activity. For example, compounds may be used in the suppression of fungal infections, as a topical antifungal agent or as a general disinfectant. These compounds are not limited to the uses listed. In therapeutic use, the compounds of this invention may be administered in the form of conventional pharmaceutical compositions appropriate for the intended use. Such compositions may be formulated as to be suitable for oral, parenteral or topical administration. The active ingredient may be combined in admixture with a nontoxic pharmaceutical carrier that may take a variety of forms depending on the form of preparation desired for administration, i.e. oral, parenteral, or topical. When a compound of the invention is employed as an anti-fungal, it may be combined with one or more pharmaceutically acceptable carriers, for example, solvents, diluents and the like, and may be administered orally in such forms as tablets, capsules, dispersible powders, granules, or suspensions containing, for example, from about 0.05 to 5% of suspending agent, syrups containing, for example, from about 10 to 50% of sugar, and elixirs containing for example, from about 20 to 50% ethanol and the like, or parenterally in the form of sterile injectable solutions or suspensions containing from about 0.05 to 5% suspending agent in an isotonic medium. Such pharmaceutical preparations may contain, for example, from about 25 to about 90% of the active ingredient in combination with the carrier, more usually between about 5% and 60% by weight.

An effective amount of compound from 0.01 mg/kg of body weight to 100.0 mg/kg of body weight should be administered one to five times per day via any typical route of administration including but not limited to oral, parenteral (including subcutaneous, intravenous, intramuscular, intrasternal injection or infusion techniques), topical or rectal, in dosage unit formulations containing conventional non-toxic pharmaceutically acceptable carriers, adjuvants and vehicles. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition of the host undergoing therapy.

Additionally, the efficacious amount of an antibiotic of the invention may be administered at a dosage and frequency without inducing side effects commonly experienced with conventional antibiotic therapy which could include hypersensitivity, neuromuscular blockade, vertigo, photosensitivity, discoloration of teeth, hematologic changes, gastrointestinal disturbances, ototoxicity, and renal, hepatic, or cardiac impairment. Further the frequency and duration of dosage may be monitored to substantially limit harmful effects to normal tissues caused by administration at or above the efficacious amount of the antibiotic of the invention.

The active compound may be administered orally as well as by intravenous, intramuscular, or subcutaneous routes. Solid carriers include starch, lactose, dicalcium phosphate, microcrystalline cellulose, sucrose and kaolin, while liquid carriers include sterile water, polyethylene glycols, non-ionic surfactants and edible oils such as corn, peanut and sesame oils, as are appropriate to the nature of the active ingredient and the particular form of administration desired. Adjuvants customarily employed in the preparation of pharmaceutical compositions may be advantageously included, such as flavoring agents, coloring agents, preserving agents, and antioxidants, for example, vitamin E, ascorbic acid, BHT and BHA. The active compound may also be administered parenterally or intraperitoneally. Solutions or suspensions of the active compound as a free base or pharmacologically acceptable salt can be prepared in glycerol, liquid, polyethylene glycols and mixtures thereof in oils. Under ordinary conditions of storage and use, these preparations contain a preservative. The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacterial and fungi. The carrier can be a solvent or dispersion medium containing, for example, water, ethanol, polyol (e.g., glycerol, propylene glycol and liquid polyethylene glycol), suitable mixtures thereof, and vegetable oil.

The invention accordingly provides a pharmaceutical composition which comprises a compound of this invention in combination or association with a pharmaceutically acceptable carrier. In particular, the present invention provides a pharmaceutical composition which comprises an efficacious amount of a compound of this invention and a pharmaceutically acceptable carrier.

The present invention further provides a method of treating fungal infections in warm-blooded animals including man, which comprises administering to the afflicted warm-blooded animals an efficacious amount of a compound or a pharmaceutical composition of a compound of the invention. The invention will be more fully described in conjunction with the following specific examples which are not to be construed as limiting the scope of the invention.

Spectral Analysis

HPLC analysis of hygroscopenes and their synthetic derivatives is conducted using a Waters Alliance model 2690 HPLC equipped with photodiode array detection. Extracts and reaction products are analyzed by reverse phase chromatography on a Supelcosil LC-18 column (4.6×250 mm, 5 μm) with a mobile phase of methanol (solvent B): water containing 5 mM ammonium acetate (solvent A). A gradient from 65% B to 90% B in 30 min at a flow rate of 0.6 mL/min is used for elution. UV detection is set at 330 nm.

LC-MS Analysis

The molecular weight of the hygroscopenes and synthetic derivatives is determined using an Hewlett Packard API-electrospray LC/MS system consisting of an HP1100 MSD instrument coupled with an HP 1100 HPLC. Compounds are resolved by reverse phase HPLC as described above at a flow rate of 0.3 mL/min. The mass spectra are recorded in the negative mode with a scan range of 300-1400 m/z. The molecular formula of hygroscopenes is obtained by high resolution mass spectra measured using a Bruker Apex-II 9.4 Tesla electrospray FT-ICR mass spectrometer. The TOF-MS/MS data is obtained using a Micromass Q-TOF instrument.

NMR Spectral Analysis

NMR spectra are recorded on Bruker AMX 400 and 500 MHz NMR instruments. ¹H and ¹³C chemical shifts are measured in parts per million relative to partially deuterated solvent peaks of MeOH-d₄ at δ 3.30 and δ 49.15 for ¹H and ¹³C NMR signals, respectively. ¹H-¹H coupling constants are measured from 1D proton spectrum given in Hertz. HSQC-COSY information is obtained using an HSQC-TOCSY pulse program with a short mixing time (12 msec.).

General Fermentation Conditions

The new antibiotics designated Hygroscopene A, Hygroscopene B and to Hygroscopene C are formed during the cultivation under controlled conditions of Streptomyces hygroscopicus ATCC29253 in a wide variety of liquid culture media. Media which are useful for the production of the hygroscopenes include an assimilable source of carbon, such as dextrin, sucrose, molasses, glycerol, etc.; an assimilable source of nitrogen, such as protein, protein hydrolysate, polypeptides, amino acids, corn steep liquor, etc. Inorganic ions and trace elements such as potassium, sodium, ammonium, calcium, sulfate, carbonate, phosphate, chloride, boron, molybdenum, copper, etc., are either added or supplied as impurities of other constituents of the media. The growth temperature range is 25° C. to 37° C. The compounds are produced over a wide range of about pH (5.5 to 8) with the most preferred range being about pH 6-7. In flask cultures, aeration is provided by vigorous shaking. In bioreactors, sterile air is sparged through the culture with further agitation provided by a mechanical impeller. An antifoam agent such as silicon oil or polypropylene glycol may be added as needed.

### EXAMPLE 1

### Fermentation

Streptomyces hygroscopicus strain ATCC29253 is cultivated in liquid culture, e.g., tryptic soy broth, at 30° C. and 200 rpm for 48 hours. This culture is inoculated into the production fermentation medium (25 ml) consisting of Pharmamedia 2%, yeast extract 0.5%, glycerol 2%, K₂HPO₄ 0.1%, KH₂PO₄ 0.1%, NaCl 0.5%, and D-glucose 2% in 250 ml Erlenmeyer flasks, which are incubated at 26° C. for 3 days with an agitation rate of 250 rpm. Analysis of 3-day fermentation methanolic extracts by LC-MS showed the presence of hygroscopene A (RT=22.6 min, m/z 1216.5), hygroscopene B (RT=23.3 min, m/z 1174.7), and hygroscopene C(RT=42.9 min, m/z 1094.7).

### EXAMPLE 2

### Isolation of Hygroscopene A, Hygroscopene B, and Hygroscopene C

In particular, because of the instability of hygroscopenes to light, acid and base, all extractions and isolations are performed at neutral conditions in subdued lighting conditions. About 2 L fermentation culture containing hygroscopenes is centrifuged and the mycelial cake is extracted with 2 L methanol with mechanical stirring for 30 min. The solids are removed by filtration and the methanol is removed by evaporation in vacuo. The concentrate (0.1 L) is diluted with 0.25 L of diethyl ether and the degreased precipitate (2.5 g) is dissolved in 100 ml of methanol for further separation by preparative reverse phase HPLC. Separation is conducted using a HP/HPV C-18 column with a gradient of methanol/water containing 0.005M ammonium acetate from 65%/35% to 90%/10% in 60 min at a flow rate of 28 mL/min. Elution of the compounds is monitored by UV detection at 380 nm. Under these conditions, Hygroscopene A elutes at 34.1 min, hygroscopene B at 35.1 min, and hygroscopene C at 48.9 min. The eluted compounds are collected into amber containers in an ice bath. After the methanol is evaporated in vacuo, the concentrated solutions (50-80 ml) are freeze-dried to afford hygroscopene A (200 mg), hygroscopene B (80 mg), and hygroscopene C (15 mg) as yellow powders.

### EXAMPLE 3

### Hygroscopene A Methyl Ester

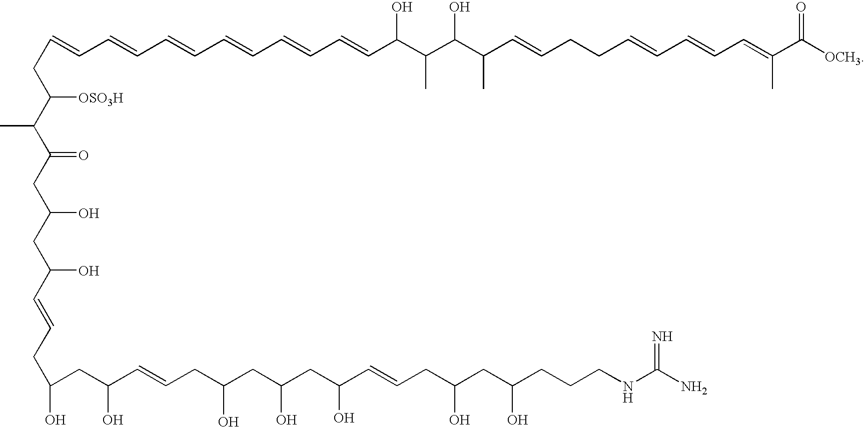

To a solution of Hygroscopene A (20 mg, 0.016 mmol) in methanol (1 ml), freshly prepared diazomethane is added at room temperature with stirring. After 30 min., the reaction is completed as indicated by the disappearance of the starting compound peak and appearance of the product peak on HPLC analysis. After solvent is evaporated in vacuo, about 20 mg of product is obtained (yield 99%).

Hygroscopene A Methyl Ester

a) Apparent Molecular Formula: C₆₄H₁₀₁N₃O₁₈S

b) ESI⁻-MS m/z 1230 [M−H]⁻ (m.w.=1231)

c) UV λₘₐₓ 305 (sh), 323, 341, 359, 380 nm

### EXAMPLE 4

### Hygroscopene B Methyl Ester

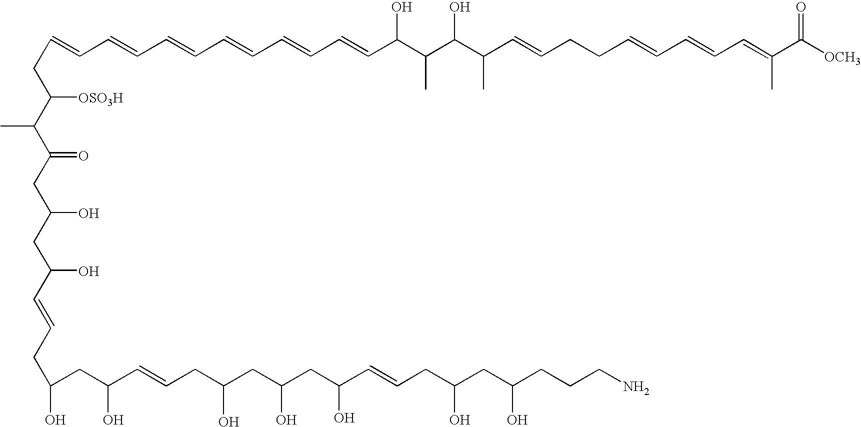

Using the conditions of Example 3 and Hygroscopene B, the product of the example is obtained.

Hygroscopene B Methyl Ester

a) Apparent Molecular Formula: C₆₃H₉₉NO₁₈S

b) ESI⁻-MS m/z 1188 [M−H]⁻ (m.w.=1189)

c) UV λₘₐₓ 305 (sh), 323, 341, 359, 380 nm

### EXAMPLE 5

### Acetyl-Hygroscopene A

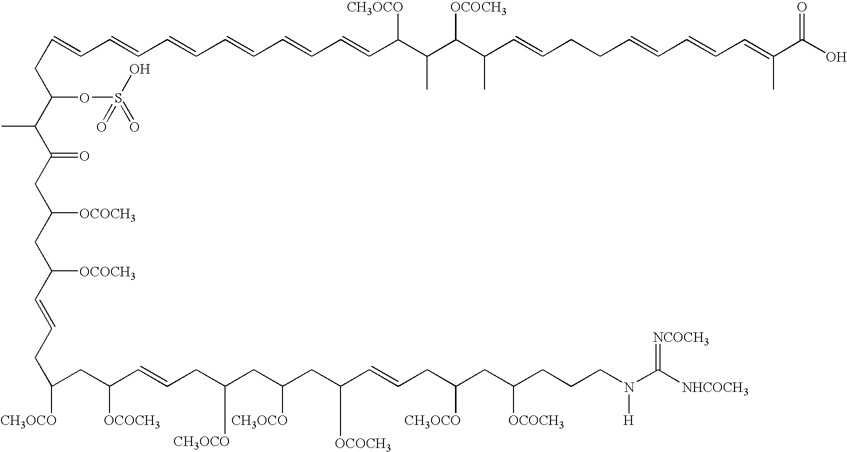

To a stirred solution of hygroscopene A (20 mg, 0.016-0.017 mmol) in pyridine (1 ml) at 0° C., acetic anhydride (0.2 ml) and 4-(N,N-dimethylamino)-pyridine (DMAP, 1 mg) is added. The reaction mixture is stirred at room temperature under nitrogen and monitored by analytical HPLC. After the reaction is completed, the mixture is dumped into 5 ml of ice water and extracted with ethyl acetate (5 ml×2). The extracts are evaporated in vacuo to yield 25 mg of Acetyl-hygroscopene A (88%).

Acetyl-hygroscopene A:_Apparent Molecular Formula: C₈₉H₁₂₅N₃O₃₁S, M.W.=1763.5

ESI⁻-MS m/z 1762 (M−H)⁻, ESI⁺-MS m/z 1786 (M+Na)⁺;

UV (MeOH) λₘₐₓ 295 (sh), 306, 323, 341, 359, 380 nm.

### EXAMPLE 6

### Acetyl-Hygroscopene B

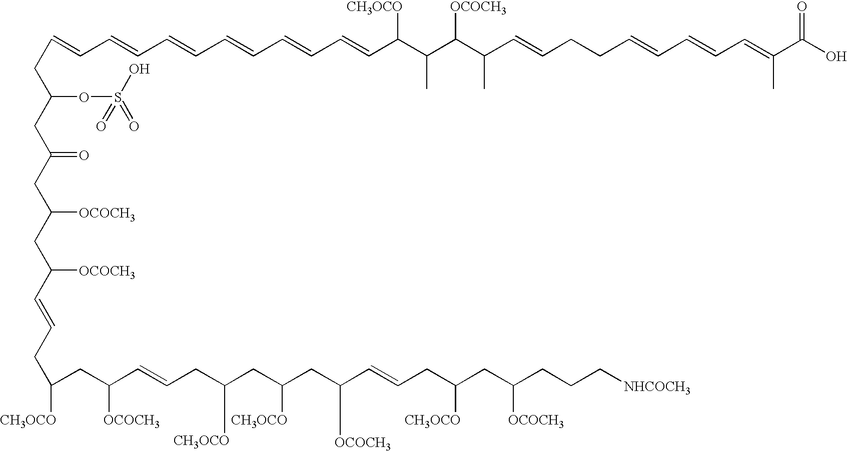

Using the conditions of Example 5 and Hygroscopene B, 24 mg of Acetyl-hygroscopene B (88%) is obtained.

Acetyl-hygroscopene B: Apparent Molecular Formula: C₈₆H₁₂₁NO₃₀S, M.W.=1679.5

ESI⁻-MS m/z 1678.6 (M−H)⁻, ESI⁺-MS m/z 1702 (M+Na)⁺;

UV (MeOH) λₘₐₓ 295 (sh), 306, 323, 341, 359, 380 nm.

What is claimed is:

1. A compound of Formula I: 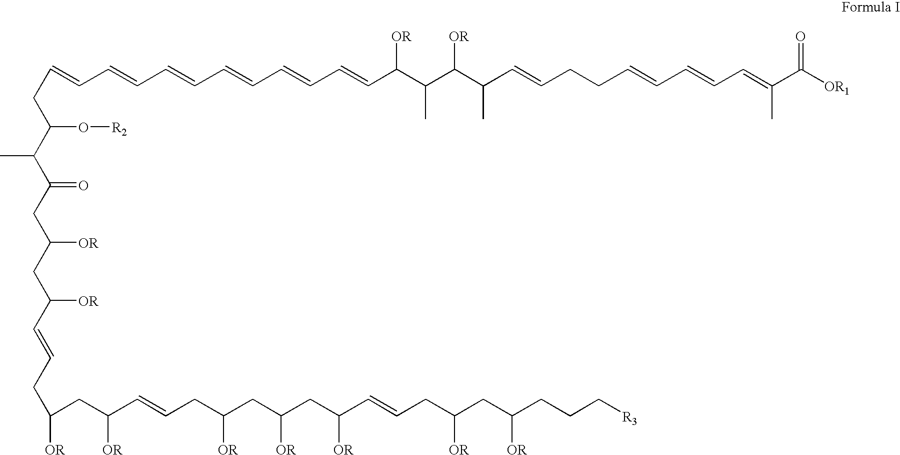
wherein:
R is H or —COCH₃;
R₁ is H or lower alkyl of 1 to 4 carbon atoms;
R₂ is H or —SO₃H;
R₃ is 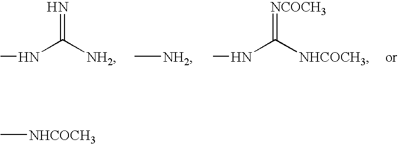
and pharmaceutically acceptable salts thereof.

2. The compound of claim 1 wherein R₂ is —SO₃H.

3. The compound according to claim 1 wherein R is H, R₁ is CH₃, R₂ is —SO₃H and R₃ is 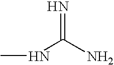
and having the structure

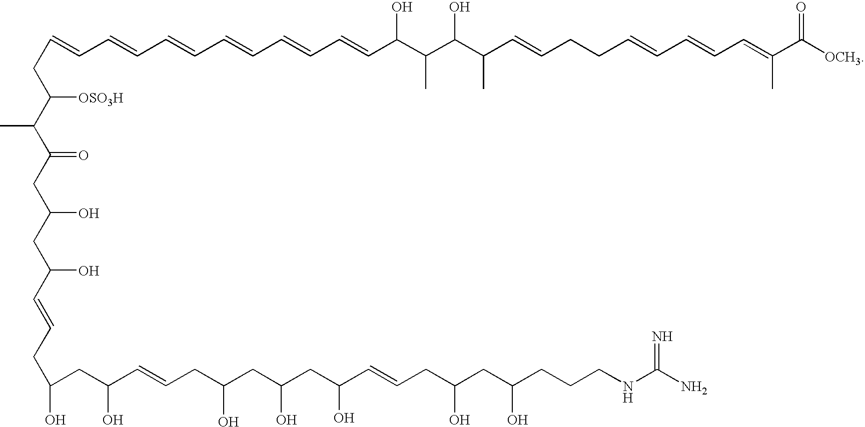

4. The compound according to claim 1 wherein R is H, R₁ is CH₃, R₂ is —SO₃H and R₃ is —NH₂ and having the structure

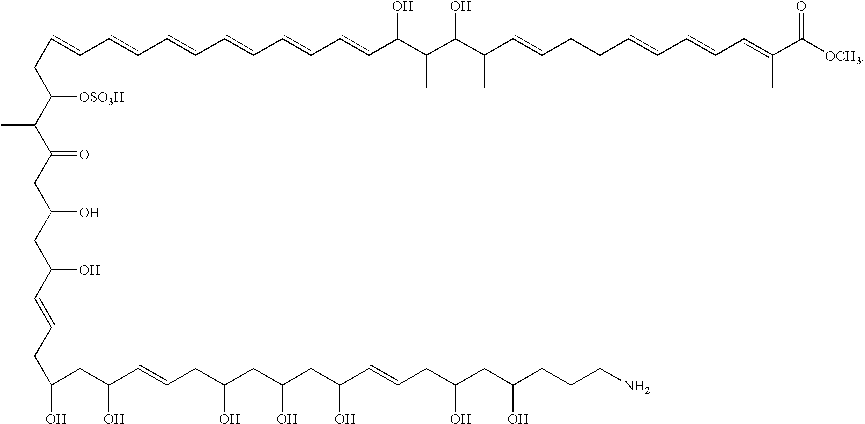

5. The compound according to claim 1 wherein R is —COCH₃, R₁ is H, R₂ is —SO₃H and R₃ is 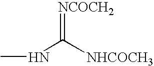
and having the structure

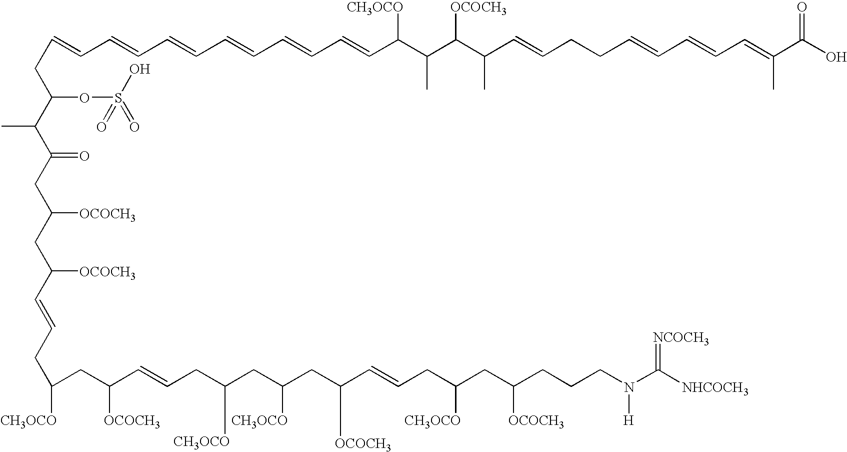

6. The compound according to claim 1 wherein R is —COCH₃, R₁ is H, R₂ is —SO₃H and R₃ is —NHCOCH₃ and having the structure

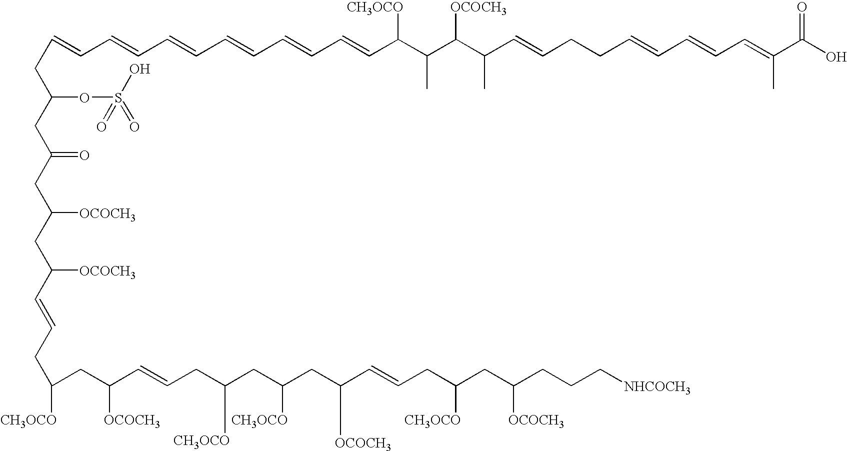

7. The compound according to claim 1 which has the structure: 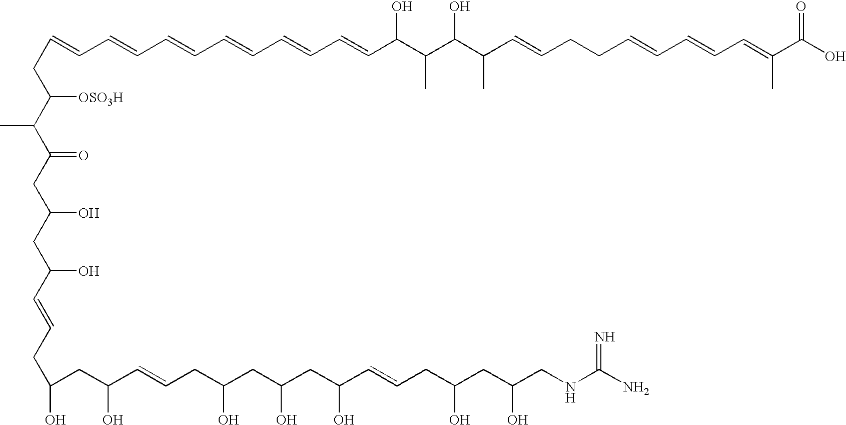
or a pharmaceutically acceptable salt thereof.

8. The compound according to claim 1 which has the structure: 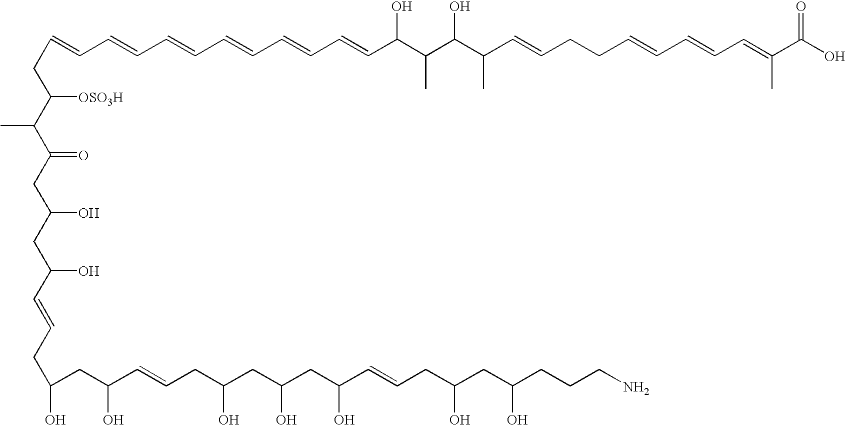
or a pharmaceutically acceptable salt thereof.

9. The compound according to claim 1 which has the structure: 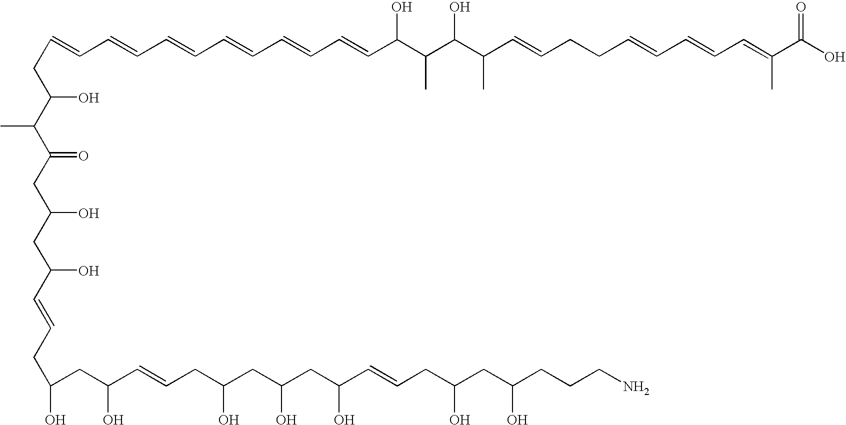
or a pharmaceutically acceptable salt thereof.

10. A method of treating a warm-blooded animal affected by fungal infections, which method comprises administering to said warm-blooded animal an effective amount of a compound of claim 1.

11. A pharmaceutical composition comprising an effective amount of a compound of claim 1 together with one or more pharmaceutically acceptable carriers.

12. A process for the preparation of antibiotics Hygroscopene A, Hygroscopene B and Hygroscopene C, which comprises cultivating Streptomyces hygroscopicus ATCC29253 or a mutant thereof under aerobic conditions, in a sterile liquid medium containing assimilable sources of carbon, nitrogen and inorganic anion and cation salts, until substantial antibiotic activity is imparted to said medium by the production of Hygroscopene A, Hygroscopene B and Hygroscopene C, recovering and isolating said antibiotics Hygroscopene A, Hygroscopene B and Hygroscopene C.

13. The process according to claim 12 further comprising separating Hygroscopene A, Hygroscopene B and Hygroscopene C into the individual components.
